(19) United States
(12) Patent Application Publication
(10) Pub. No.: US 2010/0152660 A1
(43) Pub. Date: Jun. 17, 2010

(54) CARTRIDGE WITH MULTIPLE INJECTION NEEDLES FOR A MEDICATION INJECTION DEVICE

(75) Inventors: Adam Mekeel Mack, Menlo Park, CA (US); Kenneth Alan Ritsher, Chicago, IL (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, Indianapolis, IN (US)

(21) Appl. No.: 12/600,912

(22) PCT Filed: May 22, 2008
(86) PCT No.: PCT/US08/64447

§ 371 Date: Nov. 19, 2009

Related U.S. Application Data
(60) Provisional application No. 60/940,834, filed on May 30, 2007.

(51) Int. Cl. A61M 5/32; A61M 5/20

(52) U.S. Cl. 604/136; 604/173

(57) ABSTRACT

A cartridge (260) with injection needles for an injection device. The cartridge includes a plurality of injection needle assemblies (385), each including a hub and a needle. Each needle has ends that both face in a first direction. Each hub has one portion movable relative to another hub portion between a ready arrangement and a needle assembly lifting arrangement. The cartridge includes a needle assembly support that defines a plurality of mutually parallel, needle assembly accommodating cavities in which the needle assemblies are mounted to be shiftable in the first direction from a retracted position to an injection position.

### BACKGROUND OF THE INVENTION

The present invention pertains to medical devices, and, in particular, to a cartridge with injection needles for a medication injection device.

A wide variety of medication injection devices are available which allow people, such as patients or health care professionals, to administer pharmaceuticals to themselves or others. Many of these devices are considered reusable, but utilize disposable injection needles as well as disposable medication cartridges, which medication cartridges each hold one or more doses of the desired pharmaceutical.

One type of known injection device is disclosed in International Publication Number WO 2005/097237. The device advantageously utilizes a cassette or cartridge that contains a multitude of single use injection needles, which cassette can be handled, and allows for device use, in a convenient fashion. One difficulty with this injection needles cassette is that it must be designed to function properly within the device, which device itself is complicated and subject to design limitations dictated by, for example, size and functionality constraints. And, the device and cassette interface disclosed in the above publication may not be feasible in possible alternate versions of the device.

Thus, it would be desirable to provide a cartridge for injection needles suitable for proper use within differently configured injection devices.

### BRIEF SUMMARY OF THE INVENTION

In one form thereof, the present invention provides a cartridge for a medication injection device having a reciprocating drive member. The cartridge includes: a plurality of injection needle assemblies, each injection needle assembly including a hub and a needle, each needle including a first end and a second end that both face in a first direction, each hub including a first portion and a second portion, the second hub portion movable relative to the first hub portion between a ready arrangement and a needle assembly lifting arrangement; a needle assembly support defining a plurality of mutually parallel, needle assembly accommodating cavities, each injection needle assembly mounted in a different one of the plurality of needle accommodating cavities to be shiftable in the first direction from a retracted position, at which the first and second ends both are disposed within the cavity, to an injection position, at which the first and second ends both project outside of the cavity, the needle assembly support loadable into the injection device to be movable therein to allow for separate operational alignment of each injection needle assembly with the drive member of the injection device, whereby the drive member may move to drivingly engage the hub first portion of an operationally aligned needle assembly so as to shift that needle assembly from the retracted position to the injection position; each needle assembly structured and arranged with the needle assembly support for its second hub portion to be moved relative to its first hub portion from the ready arrangement to the needle lifting arrangement upon a shifting of that needle assembly from the retracted position to the injection position, wherein for a given needle assembly in alignment for operational engagement with the drive member, the second hub portion, in a second direction opposite the first direction, is located clear of the drive member when the given needle assembly is disposed in the retracted position with the second hub in the ready arrangement; wherein for that given needle assembly, the second hub portion, in the second direction, is located adjacent the drive member when the given needle assembly has been shifted to the delivery position by the first hub portion being engaged by the drive member moving in the first direction, whereby the given needle assembly is liftable in the second direction by engagement of the second hub portion in the needle assembly lifting arrangement by the drive member when the drive member returns in the second direction.

The invention preferably is configured with the needle assembly support being a carousel rotatable within the injection device and including one of a ring of ratchet teeth and at least one pawl arm disposed radially inward of the plurality of needle assembly accommodating cavities arranged in a ring, which teeth or arm are engaged by a complimentary portion of a mounting element of the cartridge that is rotatably fixedly mountable to the medication injection device to limit carousel motion.

One advantage of the present invention is that a cartridge may be provided having needles assemblies adapted to work with a differently configured drive mechanism of an injection device.

Another advantage of the present invention is that a cartridge may be provided having needle assemblies configured so as to not interfere with a variety of ways of loading the cartridge into an injection device.

Another advantage of the present invention is that a cartridge may be provided having needle assemblies which remain properly positioned when retracted after use.

Yet another advantage of the present invention is that a cartridge may be provided which allows its needle assemblies remaining for use to be readily determined by a user.

Still another advantage of the present invention is that a cartridge may be provided for use with an injection device which allows for a more manufacturable sealing membrane configuration.

Still another advantage of the present invention is that a cartridge may be provided that is reliably mountable and indexable within an injection device.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a bottom perspective view of a first embodiment of a cartridge with injection needles of the present invention;

FIG. 2 is a top view of the cartridge with injection needles of FIG. 1, wherein portions of the cartridge, including an upper sterility-maintaining membrane, are not shown to allow further visibility of the injection needles, and wherein portions of an injection device with which the cartridge may be used are shown in dashed lines;

FIG. 3 is a cross-sectional side view of the cartridge with injection needles of FIG. 1 in an upright orientation, and wherein portions of an injection device with which the cartridge may be used are shown in dashed lines;

FIG. 4 is a cross-sectional side view similar to the view of FIG. 3 after the injection needle aligned with the injection device drive member has been driven from its retracted arrangement to its plunged, delivery arrangement;

FIG. 5 is a partial view of FIG. 2 further showing an injection needle assembly within the cartridge carousel:

FIG. 6 is a side view of the injection needle assembly of FIG. 5 removed from the cartridge carousel;

FIG. 7 is a cross-sectional view of the injection needle assembly of FIG. 6;

FIG. 8 is a partial bottom view of the cartridge of FIG. 1;

FIG. 9 is a perspective view of an alternate injection needle assembly of the present invention, wherein the drive-member-engageable tang of the hub is shown in a retracted state;

FIG. 10 is a from view of the injection needle assembly of FIG. 9, wherein the hub tang is shown in dashed lines in its retracted state, and in solid lines in its operational state;

FIG. 11 is a diagrammatic view of a carousel equipped with cavities complementarily configured for use with the injection needle assembly of FIGS. 9 and 10;

FIG. 12 is a top perspective view of another embodiment of a cartridge with injection needles of the present invention;

FIG. 13 is a cross-sectional side view of the cartridge with injection needles of FIG. 12;

FIG. 14 is a top perspective view of the carousel of the cartridge of FIG. 12 separate from the other cartridge components;

FIG. 15 is a bottom perspective view of the carousel of FIG. 14;

FIG. 16 is a top view of the carousel of FIG. 14;

FIG. 17 is a bottom view of the carousel of FIG. 14;

FIG. 18 is a top view of the pawl of the cartridge of FIG. 12 separate from the other cartridge components;

FIG. 19 is a top perspective view of the encoder disc of the cartridge of FIG. 12 separate from the other cartridge components;

FIG. 20 is a bottom perspective view of the disc of FIG. 19;

FIG. 21 is a top perspective view of the pawl retainer of the cartridge of FIG. 12 separate from the other cartridge components;

FIG. 22 is a bottom view of the retainer of FIG. 21;

FIG. 23 is a perspective view of the injection needle assembly of the cartridge of FIG. 12 separate from the other cartridge components;

FIG. 24 is another perspective view of the injection needle assembly of FIG. 23;

FIG. 25 is a side view of the injection needle assembly of FIG. 23;

FIG. 26 is a top view of the injection needle assembly of FIG. 23;

FIG. 27 is a partial perspective view of a mechanism of an injection device to which the cartridge of FIG. 12 is releasably mountable;

FIG. 28 is a partial cross-sectional view of the cartridge of FIG. 12 operationally aligned with portions of an abstractly shown injection device; and

FIG. 29 is a view similar to FIG. 28 after the drive member of the injection device has plunged the injection needle assembly for an injection.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

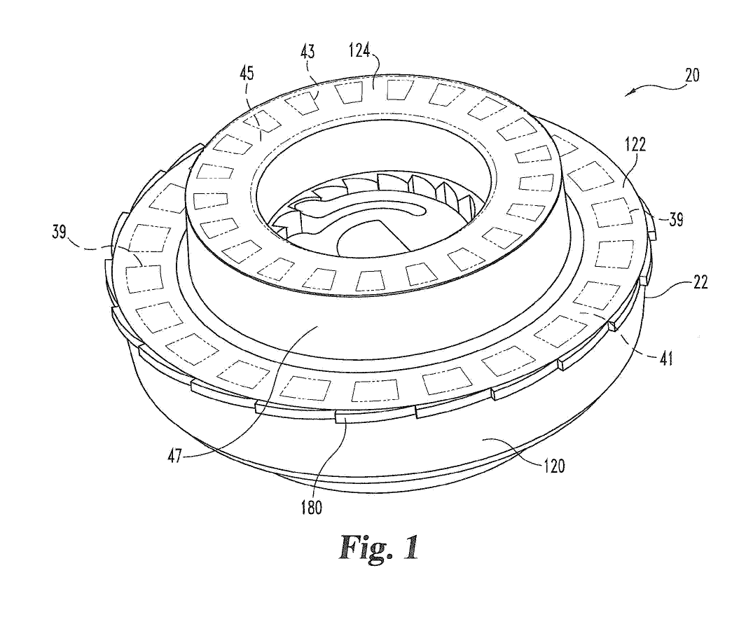

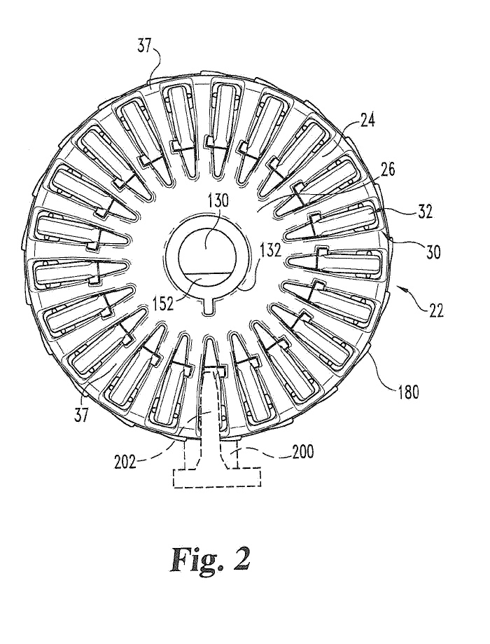

With initial reference to FIGS. 1 and 2, there is shown a first embodiment of a cartridge with injection needles of the present invention, which cartridge is generally designated 20. The cartridge 20 is particularly structured so as to be compatible with an injection device that may be similar in overall functionality, although slightly different in structure, to that disclosed in International Publication Number WO 2005/097237. The possible differences in such injection device will be apparent from the following disclosure with respect to cartridge 20. However, details of such injection device provided herein are in furtherance of an understanding of cartridge 20 and are not limiting, as the injection device itself does not form part of the inventive cartridge.

Cartridge 20 includes a support in which the injection needles are shiftably mounted. The support is shown provided in the form of a carousel 22 that is rotatable within and by the injection device when loaded therein to allow for separate operational alignment of each injection needle with a reciprocating drive member of the injection device. The axis of rotation of the carousel within the injection device is parallel to the direction in which the injection needles of the cartridge are shiftable during use. The support could be shaped differently from the round carousel shown, such as a straight, non-rotating support, if the injection device with which the cartridge is to be used requires such a configuration.

Carousel 22 includes an outer radial region 24 and an inner radial region 26. The outer radial region 24 houses the injection needles and includes a periphery allowing for a rotational indexing of the carousel within the injection device. The inner radial region 26 serves in the removable mounting of the carousel within the injection device while limiting carousel motion.

Outer radial region 24 defines a plurality of slots or cavities, generally designated 30, in each of which fits an injection needle assembly, generally designated 32. All the cavities 30 are mutually parallel, identical and are located at equal angular intervals around the circumference of radial region 24. The number of cavities and in this embodiment needles is variable by the manufacturer. The shown cartridge having twenty-one cavities that house twenty-one, single use injection needles is well suited for a cartridge needing to last one full week assuming three injections per day.

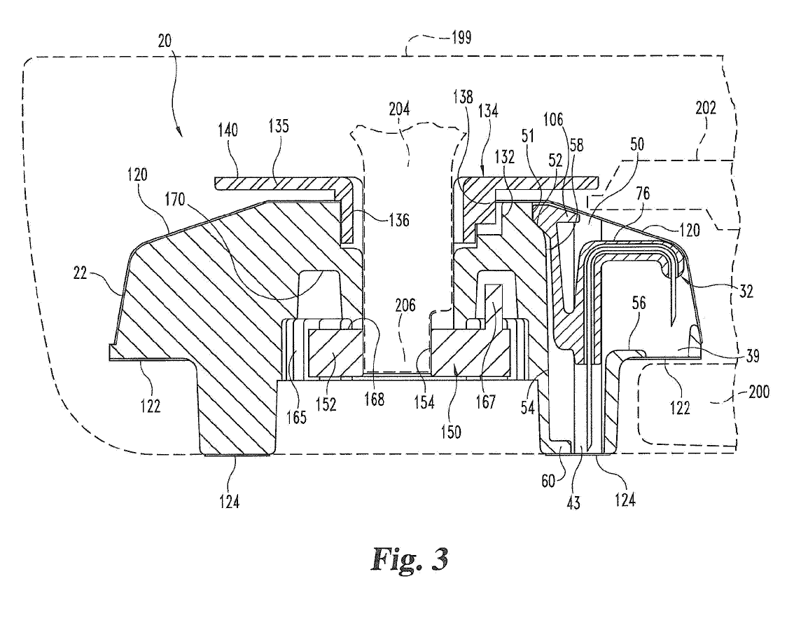

With additional reference to FIGS. 3-5, each cavity 30 has a mouth portion 35 that opens to the upwardly facing or top surface 37 of carousel region 24. Top surface 37 is shown with a multi-curved or contoured design that slopes downward as it extends radially outward. Mouth portion 35 is sized and shaped to allow operational engagement of the contained injection needle assembly by the drive member 202 of the injection device 199. For an injection device drive member that extends radially inwardly above the surface 37 and moves vertically during its use, mouth portion 35 extends downward along the radially outward extending top surface 37 sufficiently to allow for the operational range of drive member plunging motion.

Each cavity 30 also has a first port 39 that opens to the planar, annular, downwardly-facing surface portion 41 of carousel region 24, and a second port 43 that opens to a planar, annular bottom surface portion 45. Surface portions 41 and 45 are spanned their entire circumferences by a carousel wall 47 that extends in an axial direction.

First port 39 and second port 43 are sized and shaped to allow passage therethrough of inlet and outlet portions, respectively, of an injection needle. The outlet portion of the needle is the user skin-penetrating needle tip, while the inlet portion is, in the shown embodiment, the needle tip that accesses the medication within the device. The provision of such ports as separate openings within the downward facing areas of outer radial region 24 increases the area available for attachment with the sealing membrane therefor, but such ports could be provided as different regions of a common opening within the scope of the invention.

The cavity-defining surfaces of outer radial region 24 include a longitudinally or axially extending grooved region 50 along one side at a middle portion of the radial extent of the cavity, as well as a vertical region 51, an angled camming region 52, and a base region 54 at the inner radial portion of the cavity. The cavity-defining surfaces also include the upper face 56 of the carousel wall that forms surface portion 41, which face serves as a stop abutment for the injection needle.

Vertical region 51 begins at mouth portion 35 and has a lower end where it transitions to the radially inward end of camming region 52. Camming region 52 is disposed at a downward angle of forty-five degrees from vertical region 51 and transitions at its radially outer end to cavity base region 54. The upper portion 58 of base region 54 serves as a rigid support that maintains in a biased inward arrangement the needle assembly hub portion that slides therealong after being shifted inward by camming region 52 during plunging of the injection needle within that cavity. The lower portion of base region 54 is formed by a flange 60, which flange juts outward to form a capture for the needle assembly as well as provides additional surface area to which a sealing membrane is attached to the support.

The sealing of the cavities 30 to maintain sterility of the unused injection needle assemblies housed therein is provided by at least one, such as three different sealing membranes, that cover all the openings to the cavities. A first or upper sealing member 120 protectively covers the top of each cavity 50 by sealing to carousel top surface 37 around each mouth portion 35. A second or middle sealing member 122 is provided in the form of an annulus and seals to carousel surface 41 around each port 39. A third or bottom sealing member 124 is provided in the form of a smaller diameter annulus and seals to carousel surface 45 around each port 43. Different types of sealing, membranes as are known in the may be selected based on assembly and usage requirements, including consideration of factors such as toughness, coring, and effects such as flaking and removal of the silicone coating of the needle. Materials for such sealing membranes may comprise, for example, foils, or foil laminates, or polymeric films, such as polyethylene or PVC plastic.

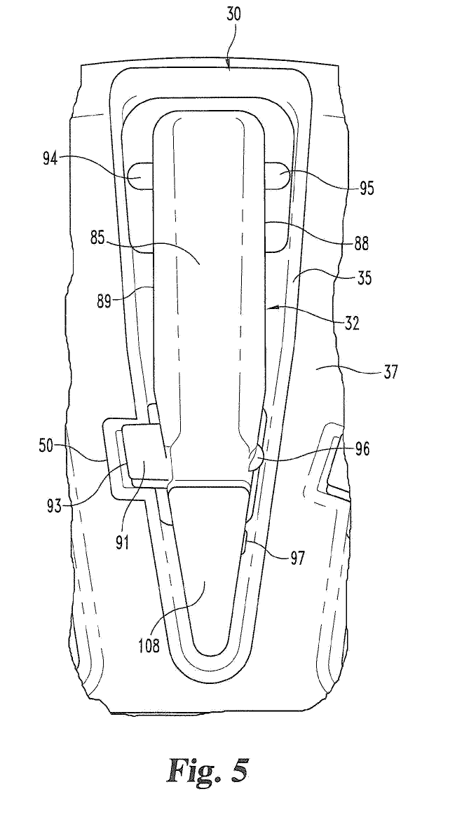

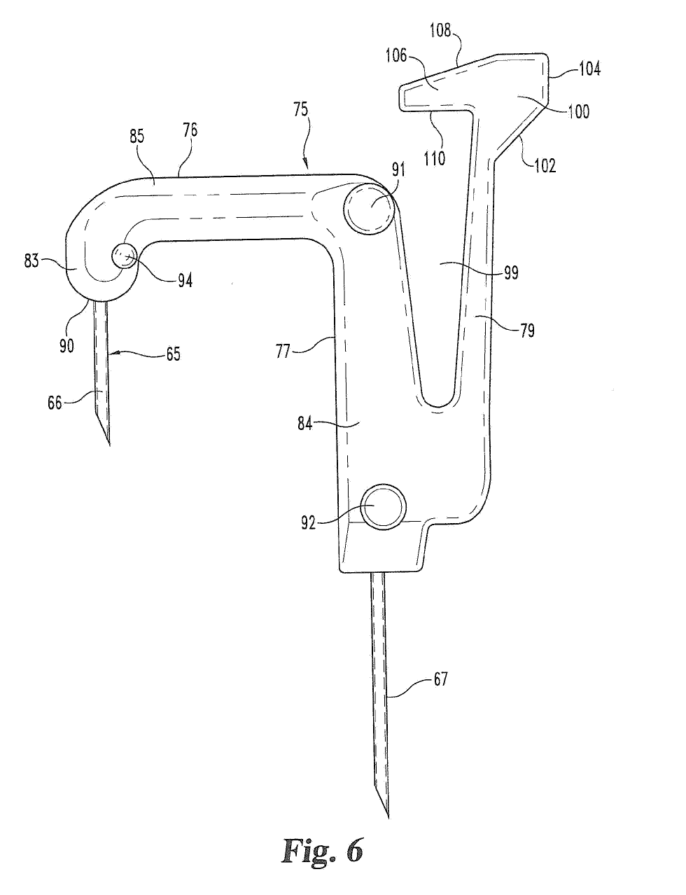

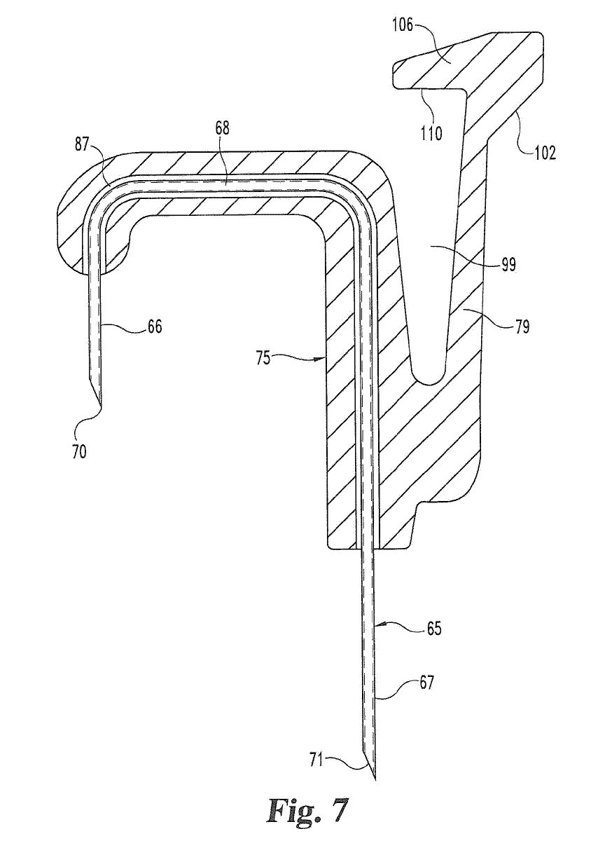

With additional reference to FIGS. 6 and 7, an injection needle assembly 32 that is axially movable relative to carousel 22 within the carousel cavity 30 during its respective operation is described. The description of needle assembly 32 is representative of all of the needle assemblies of the shown cartridge 20. Needle assembly 32 is formed of a needle or cannula 65 and a support hub 75.

Cannula 65 is generally J-shaped and includes a first leg segment 66 and a second leg segment 67 that are mutually parallel and which are fluidly connected by a spanning segment 68. The ends of needle segments 66 and 67 are shown as a septum-piercing tip 70 and a skin-piercing tip 71, respectively, that extend in the same direction, but which end at different vertical locations. The shown end of leg segment 66 being a septum-piercing tip 70 is a function of the medication container of the injection device being described herein as having a pierceable elastomeric sealing septum, which container is shown in dashed lines at 200 in FIGS. 2-4. Differently functioning cannula ends, such as a valve-accessing configuration, may be used with different medication container configurations within the scope of the invention.

Support hub 75 includes a base 77, an upstanding arm 79 and a tang 106. Base 77 is generally J-shaped and includes a shorter leg 83, a longer leg 84 and a connecting span 85. Base 77 includes a channel 87 that extends along its J-shaped length and that opens to one side 88 of the base. The channel 87 accessed through such opening receives the cannula 65 therein, which cannula is secured to base 77 within the channel by suitable means, such as an adhesive.

The lower end 90 of hub leg 83 is intended to project out of the carousel during use to meet the septum of the medication container 200. Leg lower end 90 is shown rounded in the Figures and is intended for use with sealing membranes chosen for their limited flexibility, as the rounded end 90 serves to deform such sealing membrane without further tearing.

A pair of cylindrical guide bosses 91 and 92 project from hub side 89 and each include a radially aligned outer face 93 that matches the radially aligned wall of channel or grooved region 50 of cavity 30. Bosses 91 and 92 slide within grooved region 50 and thereby guide the travel of the needle assembly 32. Bosses 91 and 92 are spaced apart along the height of the hub, and boss 91 is not aligned directly above boss 92, but rather is offset radially outward thereof. This offsetting serves to take up tilt tolerance as the bosses 91 and 92 engage opposite radial walls of the channel 50 to thereby optimize the vertical alignment of the needle cannula leg segments 66 and 67. This guiding causes needle assembly travel to be linear, in a direction parallel to the cannula segments, and such that the cannula tips penetrate perpendicularly the injection site and medication container.

The centering of support hub 75, and thereby the needle cannula 65, in the tangential direction within carousel cavity 50 is provided by, in addition to bosses 91, 92, a series of discrete contact paints in the from of nubs that project from the sides of the hub. Such nubs are shown at 94 and 95 on hub leg 83, as well as at 96 on hub 84 and at 97 on upstanding arm 79, and reduce contact and friction.

Hub arm 79 extends upward from the base of hub leg 84 at an angle so as to provide a radial gap 99 therebetween. The radially inward face of arm 79 includes contoured projection 100 at its upper end. Projection 100 includes at its lower region a ramping surface 102 that transitions to a generally vertical aligned hold surface 104 at its upper region. Ramping surface 102 and hold surface 104 are sized and shaped complementary to angled camming region 52 and vertical region 51 of cavity 30. This complementary design, and in view of the spring arm effect of the arm 79, causes the hub to fit against the cavity wall to retain the needle assembly in an upward, or non-plunged, arrangement in the carousel cavity.

The inward face of projection 100 is provided with a full radius for improved guidance during travel to limit twisting of the hub within the cavity, during which travel the hub arm, due to its resilient plastic construction, is bendable to change the size of the radial gap 99.

Tang 106 projects from hub arm 79 at its upper end and extends radially outward above gap 99. The upper face 108 of tang 106 is angled downward so as to not interfere with sealing membrane 120 covering cavity mouth portion 35. The lower surface or underside 110 of tang 106 is oriented transverse to the direction needle travel and is intended for engagement with injection device drive member 202 as described further below.

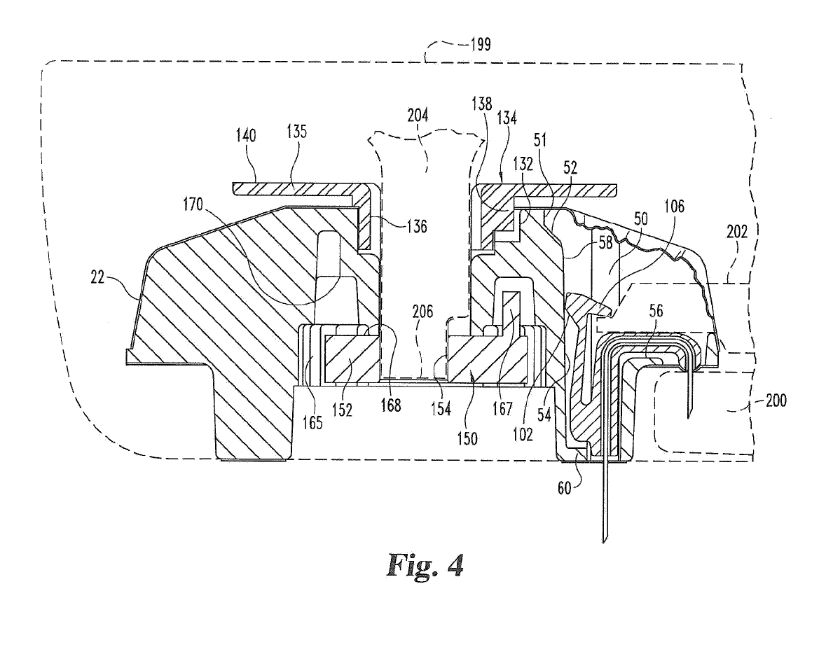

When an unused cartridge 20 is provided to a user, and also after being loaded into an injection device for use, all of the needle assemblies 32 are disposed within their respective cavities 30 in a ready or non-plunged arrangement such as best shown in FIG. 3. In this arrangement, the tang 106 of each needle assembly is clear of the space directly above the portion of its hub 76 over which the injection device drive member 202 will extend when plunging that needle assembly 32 for an injection. When the injection device loaded with cartridge 20 is operated for an injection, thereby causing the drive member 202 to shift downward, the hub portion 85 aligned under the shifting drive member is directly abutted and driven downward thereby, forcing needle assembly 32 to move downward. During this downward needle motion, resilient arm 79 pivots or bends radially outward toward hub leg 84 as ramping surface 102 slides along and is cammed radially outward by cavity wall region 52. Arm 79 is maintained outward by the engagement of hold surface 104 against cavity wall region 58 when the needle assembly has been fully plunged. The radially outward movement of bending arm 79 carries tang 106 to an adjacent position directly above, although not necessarily in contact with, the drive member 202. At this point the needle assembly 32 is arranged as shown in FIG. 4, with tang 106 being properly arranged to perform its needle assembly lifting function in association with drive member retraction.

In particular, as the injection devices reciprocates the drive member 202 so it returns upward to its ready position within the injection device at the end of an injection, the tang underside 110 is directly engaged by the upward facing surface of the drive member to effectively lift the needle assembly upward within carousel 22. The needle assembly 32 is lifted by the drive member until the tang 106, due to the hub arm resiliency, moves back radially inwardly clear of engagement with the drive member 202 as the hub arm projection region 102 reaches and slides upward along the camming region 52. This sliding upward of the hub arm along camming region 52 tends to pull the needle assembly up toward its original retracted position, such that even though the tang disengages from the drive member before the needle assembly 32 reaches its originally retracted position, the needle assembly retraction wilt continue as necessary for the needle assembly to reach, or be close to reaching, its originally retracted position, at which point the camming region and hub arm interface satisfactorily maintain the needle assembly so that the needle tips do not extend or drop down too low within the carousel to a position extending from the ports 39, 43 which would compromise further device operation.

With reference to FIGS. 2-4, the inner radial region 26 of carousel 22 defines a central hollow 130 extending the full height of the carousel. A keyed recess 132 formed in the top of carousel 22 along hollow 130 receives a cap or disc 134. Disc 134 includes a top, annular portion 135 from which depends a collar 136 with a radially projecting key 138. The keyed collar 136 seats within recess 132 and is fixedly held thereat. Top portion 135 protectively projects over the inner radial region of the needle assemblies 32 covered by the upper membrane 120.

The top surface 140 of disc portion 135 may be provided with indicia. Such indicia can convey to a user of the cartridge with an injection device a variety of information, such as the number of needles remaining to be used in the carousel. One form of indicia functions with photosensors provided in the injection device. For example, a black/white encoding system (not shown) may be provided, on top surface 140, such as a code strip with differing reflectivities arranged in a circular pattern. The code strip would be read by photosensors that are within the injection device and connected with the injection device computer. The photosensors are aligned with the code strip when the cartridge 20 is loaded in the device. In one embodiment, the code strip will be complemented by a reference strip, such as in the form of a single color strip radially inward of the code strip and that extends the complete circumference of the code strip. A single photosensor of the injection device will recognize the color or reflectivity of the reference strip for comparison against the color(s) recognized by the photosensor(s) reading the code strip. If two photosensors are provided in the injection device to read the code strip, in order to define twenty-one carousel positions, each carousel position having a unique encoder pattern, a base 5 code would be used for the code strip. The encoding system allows the carousel rotational position within the injection device to be accurately determined, allowing the injection device computer system to determine and then visibly indicate to the user on the device display the number of unused injection needles remaining in the cartridge 20. More sensors could also be used, with an appropriate black/white code.

In a not shown, alternate embodiment, the indicia may be provided in a form suitable for an electrical encoding system rather than an optical encoding system. In one such system, the black/white pattern of the indicia would be replaced with a conductive/non-conductive pattern, such as via resistive ink printed on a conductive encoder label. For a binary code, five probes or electrical contact sensors would be utilized in the injection device, plus a sixth probe to act as a common pole. Fewer probes may be used if the indicia were created with varying levels of electrical resistance, analogous to a gray-scale optical pattern.

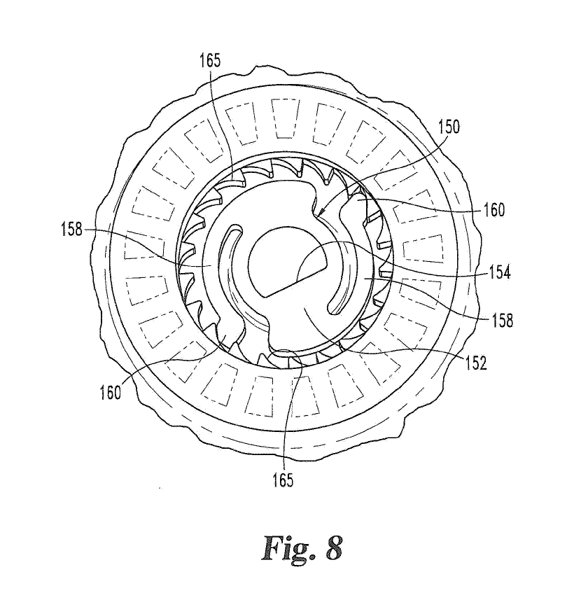

At the base of carousel 22, the central hollow 130 of inner radial region 26 is enlarged to accommodate a pawl, generally designated 150. As best seen in FIG. 3 and FIG. 8, pawl 150 includes a flattened body 152 with a keyed opening 154 centrally disposed therein. A pair of diametrically disposed, arcuate arms 158 having pawl teeth 160 extend from body 152. Teeth 160 engage a ring of one-way ratchet teeth 165 formed along the circumference of a surface of inner radial region 26. In an alternate embodiment, the positioning of the pawl arms and ratchet teeth can be switched, namely the pawl arm(s) being provided on the carousel and the ratchet teeth being provided on the pawl.

Pawl 150 also includes a locking stub 167 that upwardly projects from the top surface 168 of pawl body 152. Stub 167 fits within a cavity or channel formed by notched region 170, which channel opens to central hollow 130. Notched region 170 extends most but not all of the carousel circumference, and the end wall (not shown) of channel region 170 forms a physical stop for engagement of stub 167 as described further below which limits the rotation of the carousel 22.

As abstractly shown in dashed lines at 204 in FIGS. 3 and 4, a post associated with the injection device 199 extends through the central opening of disc 134 and within carousel hollow 130 to insert within pawl opening 154. Body keyed opening 154 is shaped to press fit with the similarly keyed bottom end portion 206 of the post to be rotatably fixed thereto. Post 204 is the axis, parallel to the injection needles, about which cartridge 20 is rotatably mounted within the injection device to allow the needles assemblies 32 to be moved into a proper angular operational position with respect to the medication container 200 and drive member 202 of the injection device.

Pawl 150 functions within the carousel inner radial region 26 to guide the angular positioning of the carousel 22 relative to the pawl 150, and therefore, via post 204, the injection device. Rotation of carousel 22 about pawl 150 and therefore post 204 is effected by an indexing system, which includes a series of teeth 180 provided on and ringing the exterior of carousel 22 against which are sequentially pushed by an advancing element of the injection device. As the carousel 22 is rotatably driven to move the next available injection needle assembly into an operational alignment with the drive member 202, pawl arms 158 resiliently bend as pawl teeth 160 ramp along and then snap over ratchet teeth 165, at which point the carousel 22 has shifted such that the next injection needle for use is angularly positioned for being independently placeable in fluid communication with medication container 200 via drive member 202. Teeth 160 and 165 prevent backward rotation of the carousel about the pawl and post. When the last injection needle 32 of the cartridge 20 has been used, the indexing system can not rotatably advance further the carousel about pawl 150 and therefore post 204 due to the abutment of locking stub 167 against the end wall of channel region 170. To prevent excessive loading on the indexing system at this stub locked arrangement, and although not shown in the Figures, the last tooth along the ring of teeth 180 may be omitted so as to not provide a surface against which the indexing element of the injection device can push.

Cartridge 20 can be employed with injection devices that provide such a post 204 in a variety of different manners for loading the cartridge into the injection device. For example, so as to allow loading of the cartridge 20 outside of the injection device housing, the post can be provided on a slide that extends from the device housing, which slide may be a part of a T-slot system, or the post can be provided on a arm that swings out as a unit from the housing, or the post can be provided on an articulating linkage that moves or swings out of the housing.

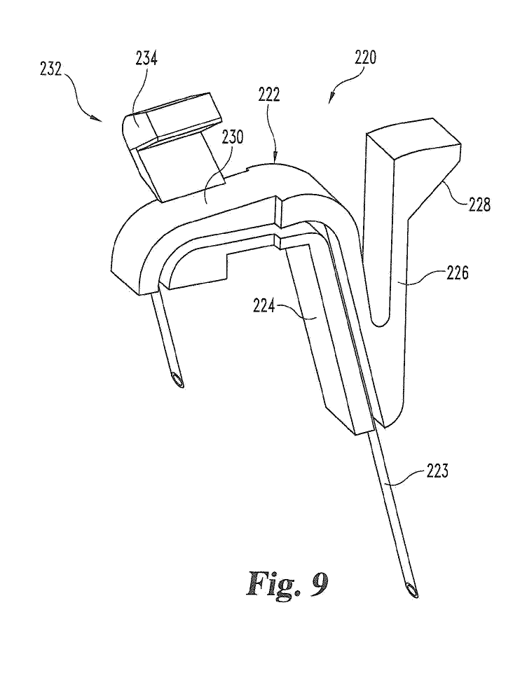

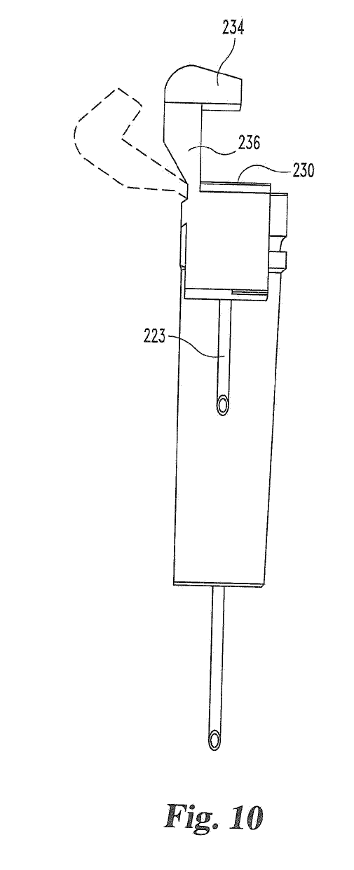

Referring now to FIGS. 9-11, there are shown portions of an alternate embodiment of a cartridge with multiple injection needles of the present invention. The shown injection needle assembly 220 includes a hub 222 with a J-shaped cannula 223 fixedly mounted to the hub body 224. Hub 222 also includes a spring arm 226 extending upwardly from the longer leg portion of hub 220. A ramp-shaped projection 228 that faces radially inward is formed on the upper end of arm 226. A spanning portion of the hub intended for engagement with the drive member of the injection device is indicated generally at 230. A tang portion 232 is formed as an orthogonal projection 234 from flange 236, which flange is pivotably attached to the hub portion 230. Such attachment, may be provided in the form of a living hinge as is known in the art. Hub tang 232 is pivotable from a retracted arrangement, shown in FIG. 9 as well as in dashed lines in FIG. 10, to an upright, operational arrangement shown in solid lines in FIG. 10. In its operational arrangement, tang projection 234 is disposed above and in vertical spaced relationship with the hub portion 230 to allow the drive member of the injection device to fit therein during plunging of the injection needle.

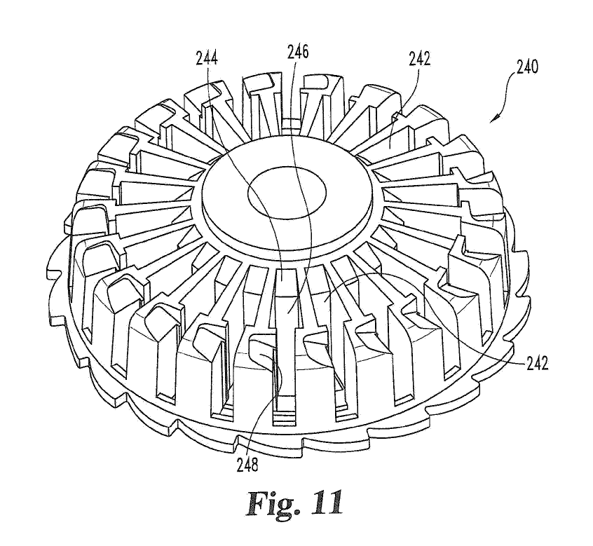

Referring now to FIG. 11, there is abstractly shown a rotatable carousel 240 provided with cavities 242, each of which cavity is suited for holding an injection needle assembly 220. Each cavity 242 includes access openings in its bottom region allowing for the needle assembly tips to penetrate the septum and the injection site similar to as performed with the embodiment of FIGS. 1-8. The radially inward portion of each cavity includes a camming surface 244 and a holding surface 246 for engagement with spring arm projection 228. Each cavity 242 thither includes a tangentially oriented, tang-camming surface 248 near a radial outward portion of the cavity. During operation, when the injection needle assembly 220 housed within such cavity 242 is plunged by an injection device drive member similar to member 200, tang 234, due to its sliding contact with the camming surface 248, pivots to the upward arrangement shown in FIG. 10 to thereby allow the injection needle 220 to be retracted by subsequent upward motion of the injection device drive member during its reciprocating return travel.

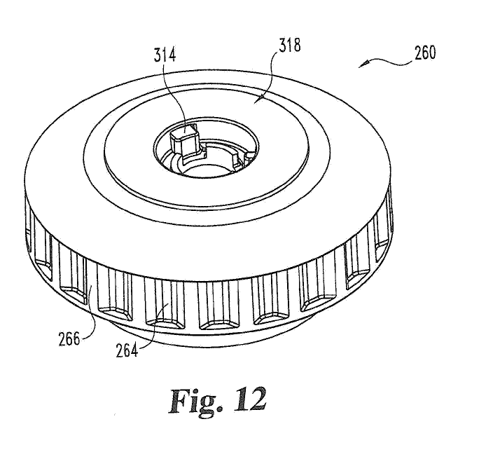

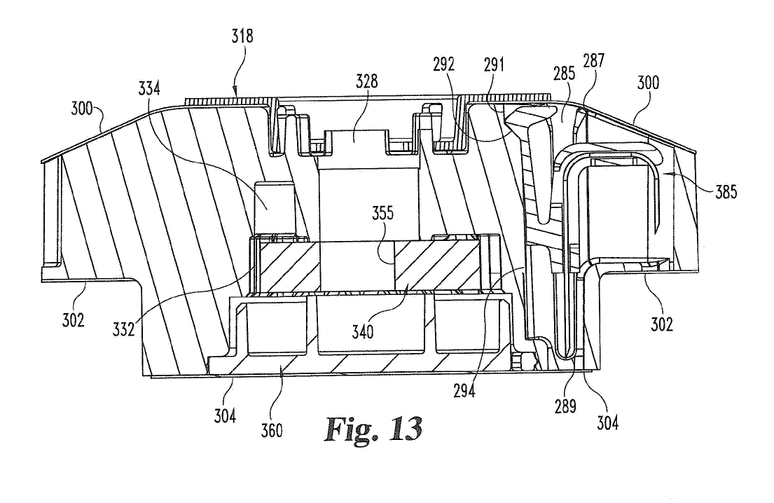

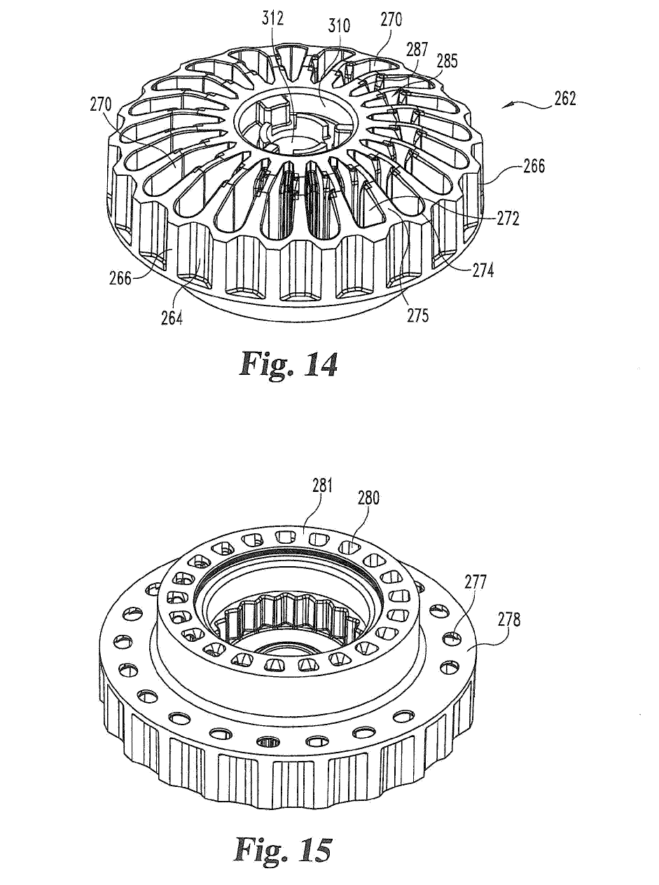

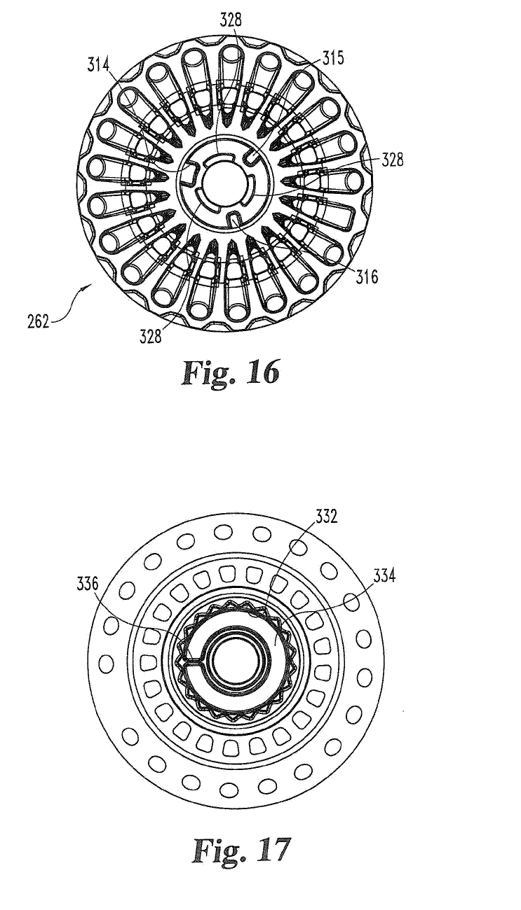

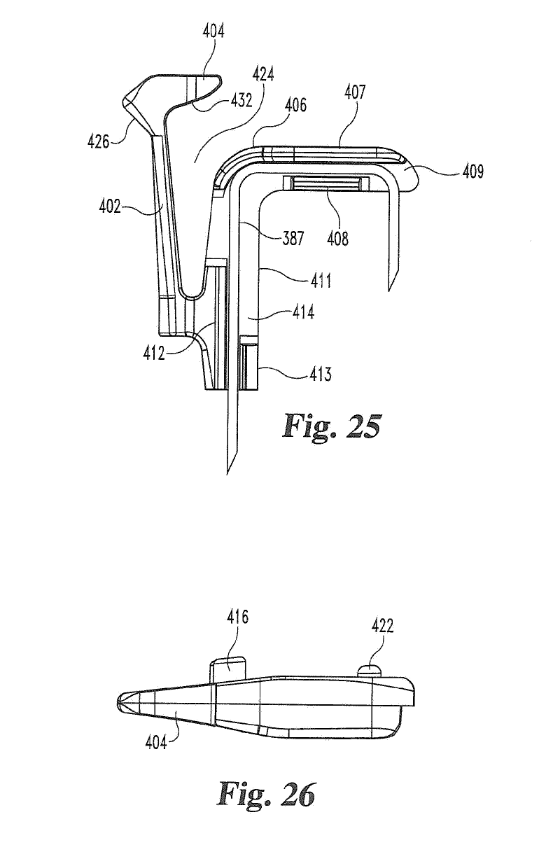

With reference now to FIGS. 12-27, there is described still another alternate embodiment of a cartridge with multiple injection needles of the present invention. The cartridge, generally designated 260, is in many respects similar to cartridge 20, but includes a series of design modifications, such as to facilitate manufacture and to operate with a differently configured injection device.

Cartridge 260 includes a needle-supporting carousel 262 molded in a single piece from ABS or other suitable material. Carousel 262 has a radial periphery provided with a series of equally angularly spaced, longitudinally extending teeth 266 formed by truncated V-shaped notches 264. The teeth 266 are provided around the entire carousel circumference. Teeth 266 are engagable by a worm gear of the injection device to rotatably index the carousel and thereby its held injection needles within the device.

In an alternate, not shown embodiment, the carousel teeth can include a gap provided by the omission of one tooth. The tooth gap aligns with the device worm gear when the last of the injection needles of the carousel has been used, which results in the carousel not being further rotatable by that worm gear thereafter.

The outer radial region of carousel 262 defines twenty identical cavities 270 and one unique cavity 272 that are evenly angularly spaced. Each of cavities 270 is intended to house an injection needle. Cavity 272 does not hold an injection needle, but rather is present, along with its fewer ports, to facilitate molding. Cavity 272 is the bay that is aligned with the drive member after the last needle assembly has been used.

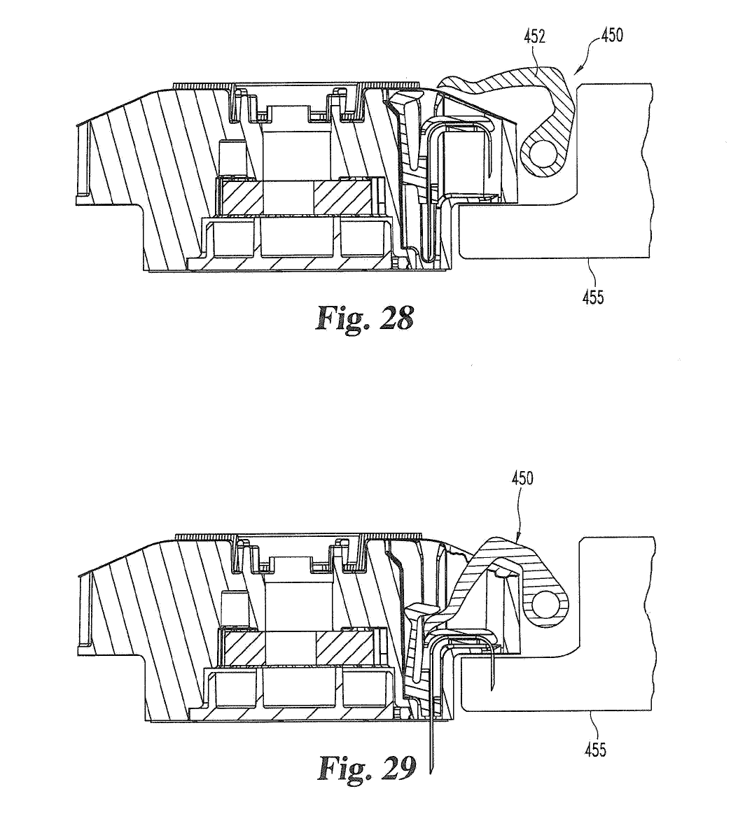

Each cavity 270 has a mouth portion 274 that opens to top surface 275. Top surface 275 does not extend as far down vertically as surface 37 of cartridge 20 due to the different injection device drive member with which it is designed to function. Rather than a drive member that reciprocates entirely vertically as shown with cartridge 20, the drive member with which cartridge 260 is particularly well adapted for use has a reciprocating pivoting motion. As further shown in FIGS. 28 and 29, wherein the drive member 450 is moved from a retracted position to a plunged position, because the drive member 450 pivots with its curved actuating arm 452 fitting over the top of the carousel side wall on which teeth 266 are formed, the cavity mouth 274 need not extend as far down, which may facilitate provision of the upper sealing membrane.

Each cavity 270 also has a first port 277 in carousel annular surface 278 for passage of an injection needle inlet portion, and a second port 280 that opens to carousel annular surface 281 for passage of an injection needle outlet portion.

The surfaces that define each cavity 270 include an axially extending grooved region 285. A chamfered top end 287 is provided for grooved region 285 to aid needle loading during assembly, and a closed, rounded bottom end 289 is provided for region 285. The cavity-defining surfaces of each cavity 270 also include a vertical region 291, an angled camming region 292 oriented at a forty-five degree angle, and a base region 294. The lower portion of base region 294 juts outward to form a stop for travel of the needle assembly.

The sealing of the cavities 270 to maintain sterility of the unused injection needle assemblies housed therein is provided by upper, middle and lower sealing members 300, 302 and 304, respectively. Upper sealing member 300 seals to top surface 275 around each mouth portion 274, middle sealing member 302 seals to carousel surface 278 around each port 277, and lower sealing member 304 seals to carousel surface 281 around each port 288 and also sealingly covers the retainer 360. Suitable sealing materials include, for example, a foil laminate for sealing member 300 which can be broken through by the injection device drive member, and a polymeric material such as TYVEK® for sealing members 303 and 304 which can be broken through by the needle assembly tips.

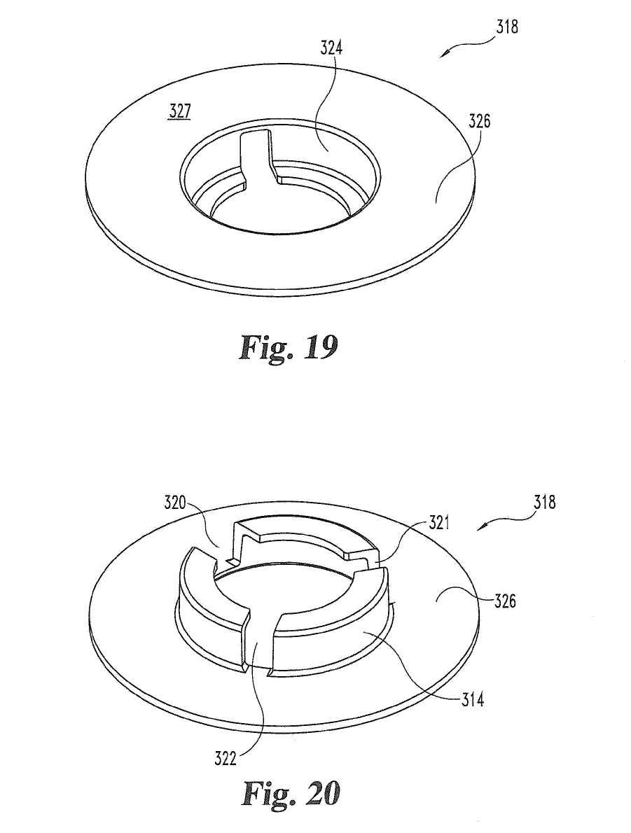

The inner radial region of carousel 262 defines a keyed recess 310 along its top surface centered on the central hollow 312 of the carousel. Keyed recess 310 is used for rotatably fixedly receiving an encoder disc, generally designated 318, further shown in FIGS. 19 and 20. Encoder disc 318 is made of ABS or other suitable material. The keying of recess 310 is provided by projections 314, 315 and 316 that fit within gaps 320, 321 and 323 provided in an L-shaped collar 334 depending from annular portion 326 of disc 318. One manner of securing disc 318 to carousel 262 is with heat staking, and sacrificial flanges used in the heat staking process are shown formed on carousel 262 at 328. The top surface 327 of disc portion 326 may be provided with indicia (not shown) described with respect to the embodiment of FIGS. 1-8, such as a gray scale encoding system printed thereon. Disc 318 radially projects within the space above the needle assemblies, but clear of interfering with the operation of drive member 450, to retain used needle assemblies after sealing membrane member 300 has been broken.

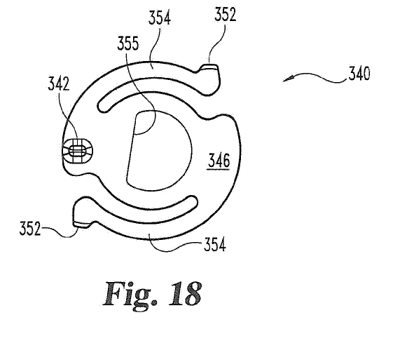

Within the lower portion of the central hollow 312, the carousel inner radial region includes a ring of one-way ratchet teeth 332. Teeth 332 function with a pawl, generally designated 340 further shown in FIG. 18. Pawl 340 is made of a sturdy but suitably resilient material, such as ABS with PTFE, or other suitable materials. Teeth 332 are engaged by pawl teeth 352 provided on the ends of diametrically disposed, arcuate pawl arms 354 to limit rotation of the carousel to a single direction, and to fixed angular increments relative to pawl 340. A locking stub 342 extending from top surface 346 of the body 344 of pawl 340 fits within a channel 334 formed in carousel 262. The circumferential length of carousel channel 334 is interrupted by a depending rib or end wall 336 that forms a physical stop for engagement of stub 342.

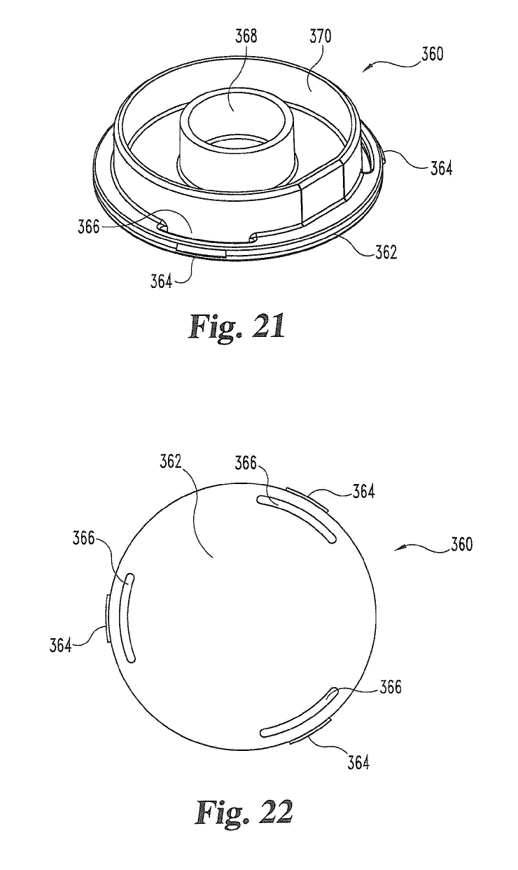

Pawl 340 is retained within the central hollow 312 of carousel 262 by a retainer, generally designated 360, further shown in FIGS. 21 and 22. Retainer 360 is molded in one piece out of ABS or other suitable materials to have a flat disc portion 362 having three detents 364 spaced around its radial periphery. An upstanding, circumferentially extending flange 368 and a shorter height, circumferential rib 370 project from the top surface of disc portion 362. Deforming recesses 366 formed through disc 362 allow deems 364 to snap lock into a grooved shoulder 338 formed at the base of carousel 262. This snap lock, assembly results in the bottom of disc portion 362 being coextensive with the bottom surface of the carousel 262, with the upstanding flange 368 supportably maintaining pawl 340 at an operational axial position within the carousel 262.

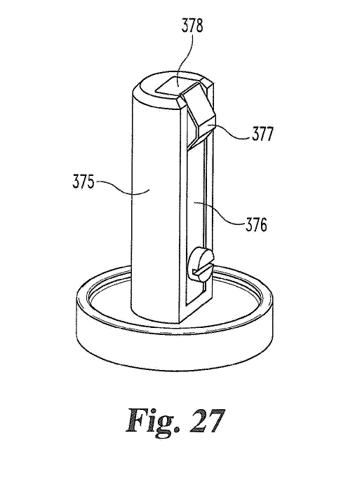

Pawl 340 includes a central keyed opening 355 through body 344 used to mown the pawl in a rotatably fixed fashion to a member of the injection device with which cartridge 260 finds useful application. One such mounting member is abstractly shown in FIG. 27 as being a post 375 with a keyed periphery sized and shaped to freely insert through the central opening of encoder disc 318 and to insert, into a rotationally fixed engagement with keyed opening 355. A spring clip 376 with a latching projection 377 is connected to the post 375 and extends along an axial channel 378 within the post 375. Clip 376 initially bends into channel 375 during the mounting of cartridge 260 to post 375 to allow projection 377 to snap over the edge of keyed opening 354 when inserted therethough and into the cylindrical space radially inward of retainer flange 368 to axially fix the pawl 340 to the post. Post 375 may be provided as part of a cartridge mounting system of an injection device such as described above with respect to the embodiment of FIGS. 1-8.

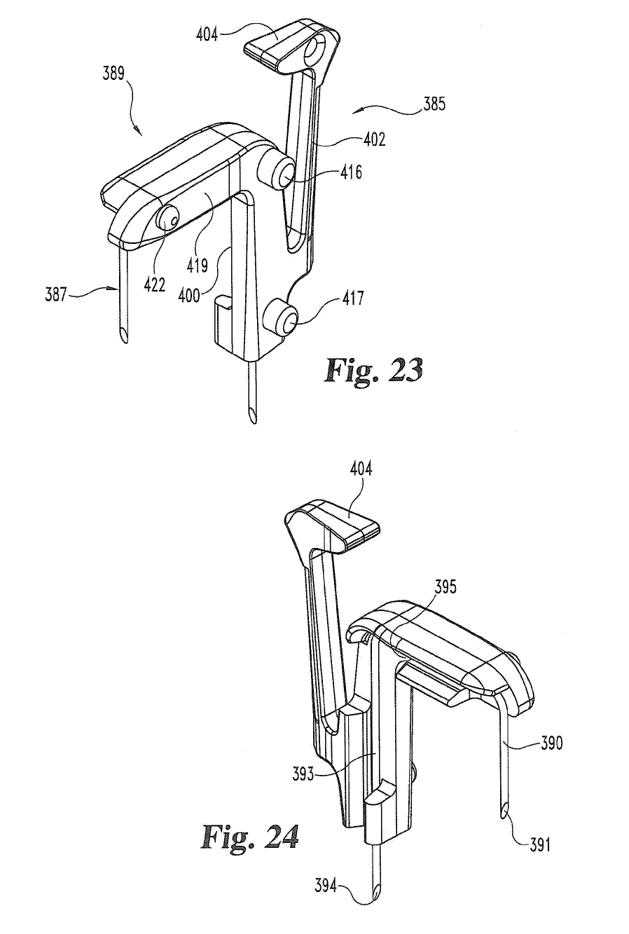

Referring now to FIGS. 23-26, a representative injection needle assembly 385 for loading and movement within any of carousel cavities 270 is shown. Needle assembly 385 is formed of a J-shaped needle 387 and a support hub 389.

Needle 387 is made of a suitable material and size for its intended use, such as stainless steel (304) and thirty-one gauge. Needle 387 is similar to needle 65 and includes a first leg segment 390 with a piercing tip 391, a parallel second leg segment 393 with a piercing tip 394, and a spanning segment 395. The piercing tips may be industry standard, such as a standard B bevel on patient end 394, and a non-coring design for the septum end 391. Cannula leg segment 393 is of sufficient length to penetrate a patient to a proper depth when used in an injection device, such as about five millimeters.

Support hub 389 is molded from plastic, such as polyethylene, in one piece with an L-shaped base 400, a resilient arm 402 and a tang 404. Needle 387 tits within the channel formed between ribs 407 and 408 that are formed on and project tangentially from the side 409 of base leg 406, as well as between ribs 412 and 413 that are formed on and project tangentially from the side 414 of base leg 411. Needle 387 is secured to hub base 400 within the channel by an adhesive.

Similarly sized guide bosses 416 and 417 project from side 419 of hub leg 411. Bosses 416 and 417 are similar to bosses 91 and 92, including having radially aligned outer faces and being offset to account for tilting. A nub 422 projecting from hub leg 406 works along with bosses 416 and 417, and to an extent the tangential facing edges of ribs 47, 408, 412 and 413, to maintain the needle tangentially aligned within cavity 270.

The angling of hub arm 402 forms a radial gap 424 with hub leg 411. A generally triangular projection from the radially inward face of arm 402 provides a ramping surface 426 that is complementary to angled camming region 292 to allow, in view of the resiliency of arm 402, the forcing of the arm 402 outward during plunging to lessen gap 424, as well as the return of the needle assembly to an upward, or non-plunged, arrangement after use as surface 426 slides up camming region 292. Tang 404 projects from hub arm 402 above gap 424. The underside 404 of tang 430 serves for engagement with the injection device drive member 450.

Needle assembly 385 is plunged within its cavity 270 by injection device drive member 450 for an injection of medication forced from medication container 455, and then withdrawn by the drive member 450 to a retracted arrangement at which the carousel is indexable for use of the next needle assembly, in a manner similar to that described with respect to the embodiment of FIGS. 1-8.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

We claim:

1. A cartridge for a medication injection device having a reciprocating drive member, the cartridge comprising:
a plurality of injection needle assemblies, each injection needle assembly including a hub and a needle, each said needle including a first end and a second end that both face in a first direction, each said hub including a first portion and a second portion, said second hub portion movable relative to said first hub portion between a ready arrangement and a needle assembly lifting arrangement;
a needle assembly support defining a plurality of mutually parallel, needle assembly accommodating cavities, each injection needle assembly mounted in a different one of said plurality of needle accommodating cavities to be shiftable in the first direction from a retracted position, at which said first and second ends both are disposed within said cavity, to an injection position, at which said first and second ends both project outside of said cavity, said needle assembly support loadable into the injection device to be movable therein to allow for separate operational alignment of each injection needle assembly with the drive member of the injection device, whereby the drive member may move to drivingly engage the hub first portion of an operationally aligned needle assembly so as to shift that needle assembly from the retracted position to the injection position;
each said needle assembly structured and arranged with said needle assembly support for its second hub portion to be moved relative to its first hub portion from said ready arrangement to said needle lifting arrangement upon a shifting of that needle assembly from the retracted position to the injection position;
in for a given needle assembly in alignment for operational engagement with the drive member, said second hub portion, in a second direction opposite the first direction, is located clear of the drive member when said given needle assembly is disposed in the retracted position with said second hub portion in the ready arrangement;
wherein for that given needle assembly, said second hub portion, in the second direction, is located adjacent the drive member when said given needle assembly has been shifted to said delivery position by said first hub portion being engaged by the drive member moving in the first direction, whereby said given needle assembly is liftable in said second direction by engagement of said second hub portion in the needle assembly lifting arrangement by the drive member when the drive member returns in the second direction;
wherein said needle assembly support comprises a carousel rotatable within the injection device and including one of a ring of ratchet teeth and at least one pawl arm disposed radially inward of said plurality of needle assembly accommodating cavities arranged in a ring; and,
a mounting element rotatably fixedly mountable to the medication injection device and including the other of said ring of ratchet teeth and at least one pawl arm to limit carousel motion.

2. The cartridge of claim 1 wherein each hub includes a spring arm, and wherein each said second huh portion comprises a tang that extends from its associated spring arm that biases said second hub portion tang toward a position clear in the second direction from said first hub portion.

3. The cartridge of claim 1 wherein said mounting element includes the at least one pawl arm and further comprises a keyed central opening sized and shaped to rotatably fixedly receive a post of the medication injection device.

4. The cartridge of claim 2 wherein each said tang extends radially outward from its associated spring arm, wherein a radially inward face of each spring arm includes a cammable surface, said needle assembly support includes a plurality of camming surfaces that each define a portion of a different one of said plurality of needle accommodating cavities, whereby for each needle assembly when shifting from the retracted position to the injection position, engagement of its cammable surface by one of the camming surfaces moves its second hub portion relative to its first hub portion from said ready arrangement to said needle lifting arrangement.

5. The cartridge of claim 1 wherein each hub includes a spring arm with a surface structured and arranged with said needle assembly support to aid in shifting its delivery needle assembly to the retracted position from the delivery position.

6. The cartridge of claim 5 wherein said needle assembly support comprises a rotatable carousel, and wherein each said spring arm surface comprises a radially inward facing ramp-shaped protuberance.

7. The cartridge of claim 1 wherein said hub of each said needle assembly, and a surface of said needle support assembly within each said needle assembly accommodating cavities, together further comprise a guide channel and a complementary projecting guide slidable within said channel for guiding motion of said needle assembly within said needle support assembly.
